(19) United States
(12) Patent Application Publication
(10) Pub. No.: US 2018/0015247 A1
(43) Pub. Date: Jan. 18, 2018

(54) Nasal Interface for CPAP Device

(71) Applicants: Stephen Alan Marsh, Carlisle, MA (US); James M. Ormond, Belmont, MA (US); Bryan R. Hotaling, Harvard, MA (US); John A. MacNeill, Acton, MA (US)

(72) Inventors: Stephen Alan Marsh, Carlisle, MA (US); James M. Ormond, Belmont, MA (US); Bryan R. Hotaling, Harvard, MA (US); John A. MacNeill, Acton, MA (US)

(73) Assignee: Encite LLC, Burlington, MA (US)

(21) Appl. No.: 15/648,504

(22) Filed: Jul. 13, 2017

Related U.S. Application Data
(60) Provisional application No. 62/362,697, filed on Jul. 15, 2016.

(51) Int. Cl. A61M 16/06; A61M 16/00

(52) U.S. Cl. CPC A61M 16/0666; A61M 16/0057; A61M 16/0616; A61M 2210/0618; A61M 2205/0216

(57) ABSTRACT

A nasal interface for a device that attached to a user's nose is described. The nasal interface is particularly adaptable for a CPAP device and includes a device body having air passages through the body terminating in a pair of end portions, with each end portion having at least one outlet in a first surface of the end portion and a nasal interface supported on the body, the nasal interface comprising a pair of nose buds having flared fittings at end portions thereof.

This application claims priority under 35 U.S.C. §119 to U.S. Provisional Patent Application Ser. No. 62/362,697, filed Jul. 15, 2016, and entitled “Nasal Interface CPAP Device”, the entire contents of which is hereby incorporated by reference.

### BACKGROUND

This specification relates to CPAP devices for treating breathing disorders.

A continuous positive airway pressure device (CPAP) device is a device for treatment of breathing disorders by supplying slight amounts of air pressure to a user to keep a user's airways open. CPAP devices are typically used by persons who have breathing disorders, such as sleep apnea. Conventionally, a CPAP machine is used and includes a mask or other device that fits over a user's nose and/or nose and mouth, straps keep the mask in place while being worn by the user and a tube that connects the mask to the machine that blows air into the tube.

### SUMMARY

According to an aspect, an airway pressure breathing device includes a body having a pair of air passages through the body, which pair of air passages terminate in a pair of end portions, with each end portion having at least one outlet in a first surface of the end portion, a micro pump supported by the body, the micro pump configured to pump ambient air through the air passages in the body to the end portions, and a nasal interface supported on the body, the nasal interface comprising a pair of nose buds having flared fittings at end portions thereof.

According to an aspect, a device attachable to a user's nose includes a body having a pair of air passages through the body, which pair of air passages terminate in a pair of end portions, with each end portion having at least one outlet in a first surface of the end portion and a nasal interface attached to the body, the nasal interface comprising a pair of nose buds, each nose bud having an external mesa shaped sidewall that terminates at a first end in a flared portion and terminates at the second end at a corresponding one of the pair of air passages of the body.

According to an aspect, a nasal interface to attach a device to a user's nose includes a pair of nose buds each having a sidewall that encloses a passageway, an external shape of the sidewall being mesa-shaped, with the sidewall terminated at one end in a flared fitting and with each of the nose buds having at a second distal opposing end of the sidewall, an accommodation to accept a device.

According to an aspect, a nasal interface to attach a device to a user's nose includes a nose bud having a sidewall that encloses a passageway, an external shape of the sidewall being somewhat mesa in shape, with the sidewall terminated at one end in a flared fitting.

One or more of the above aspects may include one or more of the following features.

The nose buds of the interface have nose bud exterior sidewalls, with the flared fittings integrally provided on the nose bud sidewalls, and the flared fittings sized to allow the user to easily insert the nose buds into the user's nose. The nose buds of the interface provide a seal between the airway pressure breathing device and the user's nose. The flared fittings have at least one aperture that connects to air pathways in the airway pressure breathing device. The nose buds of the interface have exterior sidewalls having an external mesa shape, with the flared fittings being extensions of the nose bud sidewalls. The nose buds have exterior sidewalls having an external mesa shape, with the flared fittings being extensions of the nose bud sidewalls, and with the flared fittings having an internal passage way between end portions thereof that is conical-shaped, with a first one of the end portions at the flared portions having a larger opening than a second distal one of the end portions terminating at the airway pressure breathing device. The material of the nose buds is a supple compliant soft durometer material. The nose buds have a nose bud estimated centerline angle range of about 110 to 150 degrees, a nose bud estimated face angle range of 30-50 degrees and the nose bud shapes are generally oblong. The nose buds having openings that are oblong and can range in area from about 30-90 mm². The material of the nose buds is a rubber material.

Each nose bud sidewall and each corresponding flared fitting of the interface is integrally provided with the nose bud sidewalls, and with the flared fittings sized to allow the user to easily insert the nose buds into the user's nose. The nose buds of the interface provide a seal between the device and the user's nose. The material of the nose buds is a supple compliant soft durometer material, and with the interface having a nose bud estimated centerline angle range of about 110 to 150 degrees, a nose bud estimated face angle range of 30-50 degrees and the nose bud external sidewall and flared fittings have a shape that is generally mesa shaped. The nose buds have openings there through that are generally oblong and are in a range in area from about 30-90 mm².

The nose buds of the interface each have an internal passage between the flared fitting and the second distal opposing end provided by an internal conical shaped sidewall. The nose buds provide a seal between a device to which the interface is attached and a nostril of the user's nose. The material of the nose buds is a supple compliant soft durometer material, and the interface has a nose bud centerline angle range of about 110 to 150 degrees and a nose bud face angle range of 30-50 degrees. The flared fittings of each nose bud has an opening to the corresponding passageway that is oblong shaped and ranges in area from 30-90 mm².

The nose bud of the interface has at a second distal opposing end of the sidewall an accommodation to accept a device. The material of the nose bud is a supple compliant soft durometer material and with the interface having a nose bud estimated centerline angle range of about 110 to 150 degrees, a nose bud estimated face angle range of 30-50 degrees and the nose bud having the passage way being a somewhat funnel shape. The interface includes two of the nose buds attached to a device and with each having openings to the passageway that are oblong shaped and range in surface area from about 30-90 mm².

One or more aspects may include one or more of the following advantages.

This application deals with details of an nose to device interface for an airway pressure breathing device, as generally described in US published patent application US-2015-0267695-A1, assigned to the assignee of the present invention and incorporated herein by reference. The interface holds a user's nostrils open, and possible assists with holding air passages open as well. Articulated nose buds have air passages or apertures and are made of a generally rubbery material that makes a tight fit when inserted into a user's nostrils. The nose buds can provide ease of insertion into the nostrils, along with a tight seal to the nostrils. The flare/flange fittings allow for easy insertion and comfortable positioning inside nostril cavities to provide a sealing interface to a user's nostrils. The interior shape of channels provide an air path from wide (at the portion inserted into the nostrils to narrow at the portion affixed to the body, which can reduce air flow impedance during exhalation. That is, the cross-sectional area of the opening at the end of the channels and hence volume of space provides a gradual taper to minimize resistance during exhalation. Moreover this funnel shape surface can be a smooth surface to further minimize resistance.

The details of one or more embodiments of the invention are set forth in the accompanying drawings and the description below. Other features, objects, and advantages of the invention are apparent from the description and drawings, and from the claims.

### DESCRIPTION OF DRAWINGS

FIGS. 1-8 are various views an airway pressure breathing device having an articulated nasal interface.

FIG. 1A is a diagrammatical cross-sectional view of FIG. 1 showing details.

### DETAILED DESCRIPTION

### Overview

This application deals with details of a nose to device interface for an airway pressure breathing device, as generally described in US published patent application US-2015-0267695-A1, assigned to the assignee of the present invention and incorporated herein by reference. Described in the above application is a novel micro pump that comprises plural stacks of compartmentalized pump chambers, and which uses less material, is subject to less stress, and is driven using less power than conventional pumps. The micro pump has a size in the micron to millimeter scale, and can provide wide ranges of flow rates and pressure and is part of the described CPAP device mentioned in the above application.

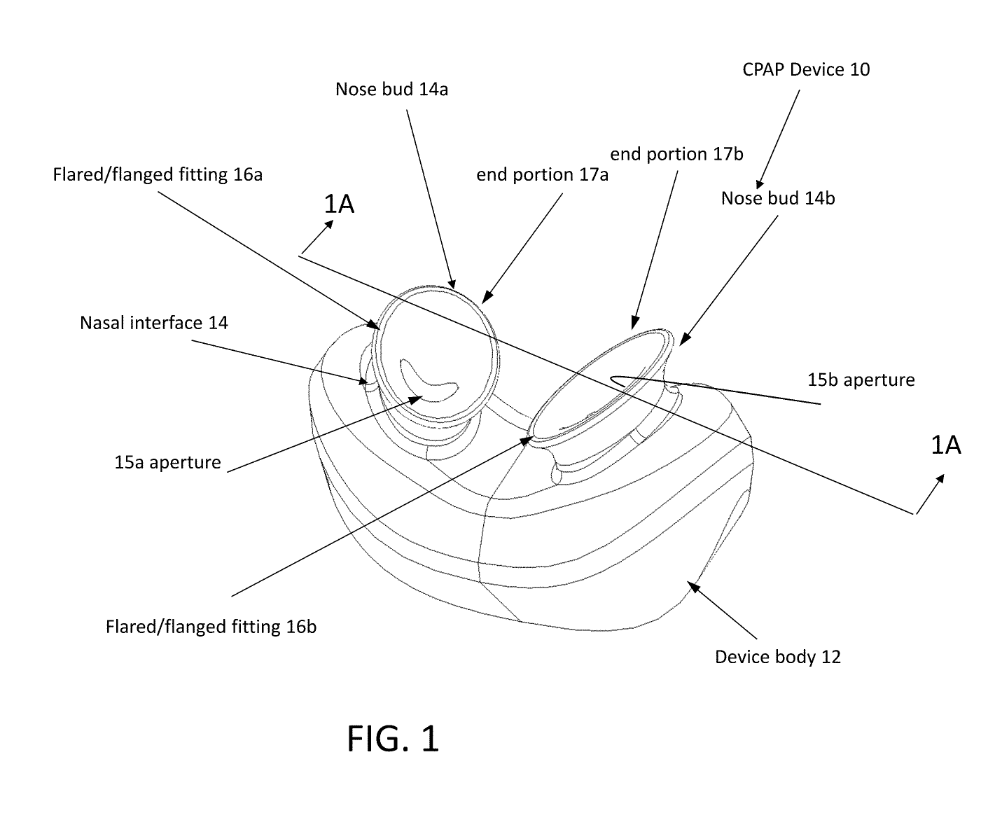

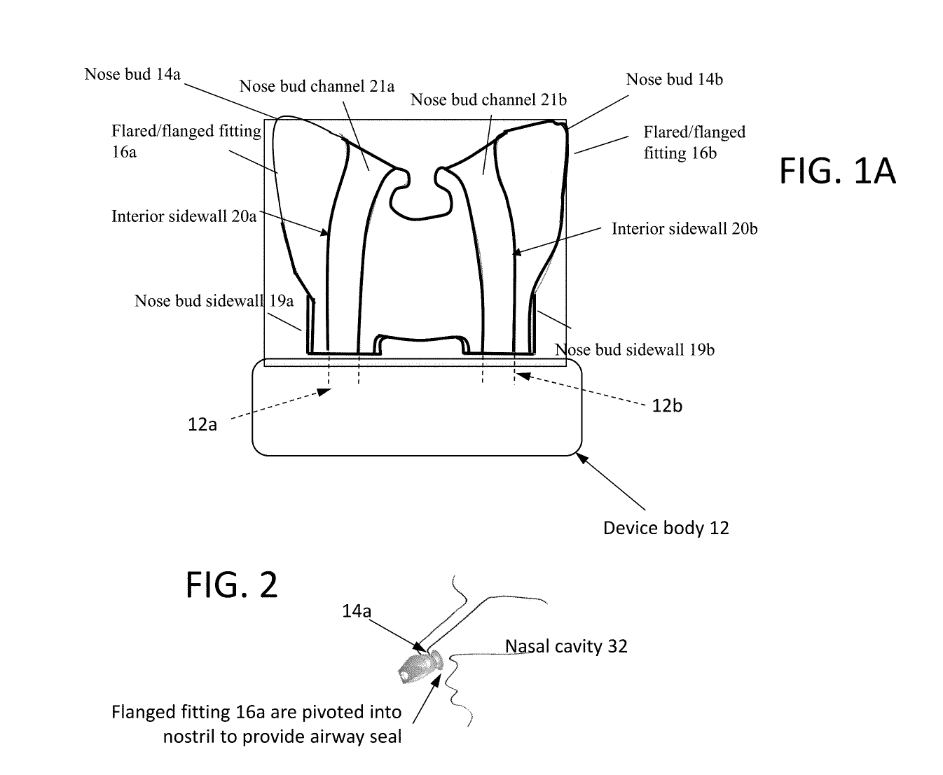

Referring now to FIGS. 1-7 an autonomous device 10 (device 10) for treating breathing disorders is shown. The device 10 is in some implementations a CPAP type (continuous positive airway pressure) breathing device 10. However, the device 10, unlike CPAP machines, is an autonomous device that is local to the nose and which provides a required amount of air flow at a required pressure to treat various breathing disorders such as obstructive sleep apnea (“OSA”), as discussed in the above mentioned patent application. In other implementations, without the micro pump and other components of the CPAP breathing device 10, the package of such device 10 can be used to hold nostrils open and possible assist with holding air passages open.

When used as the CPAP device 10, the device 10 includes a body 12 that houses a micro pump (not shown) component, an exhalation valve (not shown) and a nasal interface 14 comprised of cushioned. articulated nose buds 14a, 14b having air passages or apertures 15a, 15b through the nose buds 14a, 14b. The cushioned nose buds are made of a generally rubbery material that make a tight fit when inserted into a user's nostrils.

The nose buds 14a, 14b are attached to the breathing device body 12 through an accommodation that can vary in character, and provide the interface to the user's nostrils. While the nose buds perform a primary function of keeping the breathing device 10 in the proper place for treatment. The nose buds 14a, 14b provide ease of insertion into the nostrils, along with a tight seal to the nostrils. Examples of accommodations include a port that is received by the body or merely an aperture in the body and the interface is affixed to the body.

Referring now to FIG. 1, the nose buds 14a, 14b and their attachment to the breathing device body 12 is shown. The nose buds 14a, 14b have flare/flange fittings 16a, 16b at end portions 17a, 17b thereof, as shown. The flare/flange fittings 16a, 16b are configured for easy insertion and comfortable positioning inside nostril cavities to provide a sealing interface to a user's nostrils. The sealed interface with nostril cavity allows unobstructed breathing. The flare/flange fittings 16a, 16b are integrally provided (e.g., formed) on nose bud sidewalls, and are sized to allow the user to easily insert the nose buds into the user's nose, preferably with one hand, without excessive adjusting motion. The nose buds provide a seal between the device 10 and the user's nose. Within the flare/flange fittings 16a, 16b are at least one aperture that connects to air pathways (not shown) in the device 10.

As shown in FIG. 1A, the nose buds 14a, 14b have flare/flange fittings 16a, 16b at end portions and external sidewall 19a, 19b have a generally mesa external shape to secure fit to and affix to a user's nostrils, whereas interior sidewalls 20a, 20b of the nose bud are somewhat conical or funnel shaped forming nose bud channels 21a, 21b. Also shown diagrammatically is the body 12 of the CPAP device that would include a micro pump (not shown) and which has a pair of passages 12a, 12b that couple to the nose bud channels 21a, 21b.

The interior sidewalls 20a, 20b having one terminus at one end that is affixed to the CPAP body 12 having a relatively narrow opening into the CPAP body 12 and have a terminus at a second, distal end of the interior sidewalls having a relatively wider opening. The interior shape of the channels 21a, 21b provide an air path from wide (at the portion inserted into the nostrils to narrow at the portion affixed to the CPAP body 12, which can reduce air flow impedance during exhalation. That is, the cross-sectional area of the opening at the end of the channels 21a, 21b and hence volume of space provides a gradual taper to minimize resistance during exhalation. Moreover this funnel shape surface can be a smooth surface to further minimize resistance.

The seal allows the device 10 to build pressure in a volume that is the combination of the device body's 12 internal working volume and the user's airway path volume. The seal can be effective well above pressure limits of CPAP (continuous positive airway pressure) treatment levels (less than 20 cmH₂O) to provide ample pressure margin. It is envisioned that the seal can be effective to at least 34 cmH₂O.

The shape and size of the nose buds 14a, 14b are comfortable enough to allow the user to fall asleep. The amount of material protruding into the nose is minimal. The material of the nose buds is a very supple and conformable material to rest comfortably within the individual's nose. The nose buds 14a, 14b allow the breathing device 10 to stay in place in the nose during any incidental rubbing, turning or any other forces acting against it for the normal duration of the sleep period.

Referring now to FIG. 2, illustrated is an insertion process to install the breathing device 10 into the user's nose 30. The nose buds (14a shown) are inserted into the user's nose 30. Flanged fittings (16a shown) are pivoted into the nostrils to provide an airway seal to the nasal cavity 32, as shown. In one implementation the flanged fittings one each of the nose buds are inserted by pivoting with a upwards motion, the nose buds into the nose towards the nasal cavity to provide a reasonable tight seal between the nasal cavity and the nasal interface.

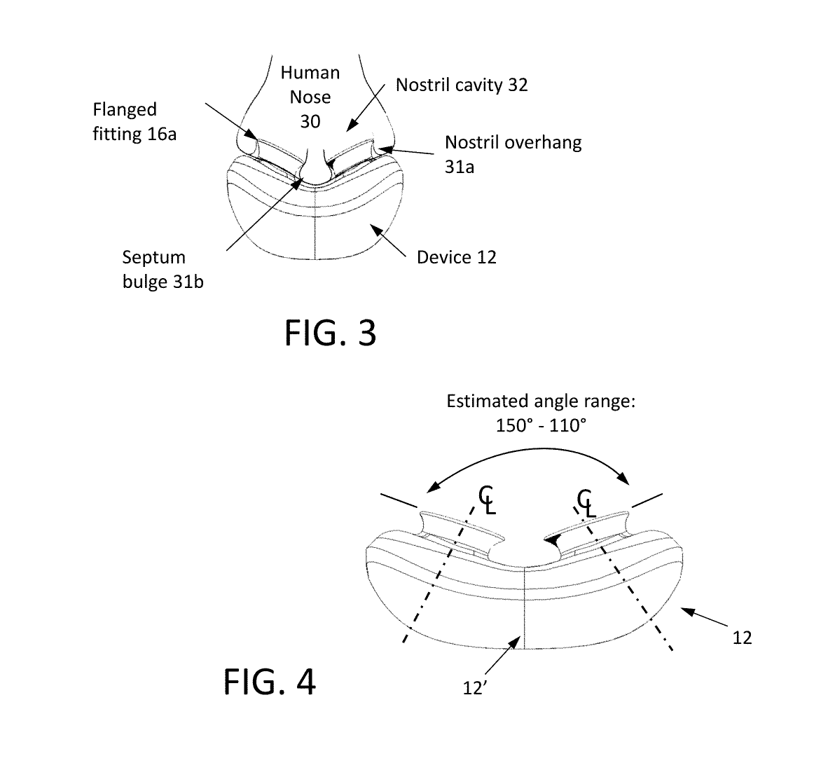

Referring now to FIG. 3, illustrates how the nose buds (not referenced) fit into the human nose 30. The flared/flanged fittings 16a, 16b of the nose buds form a seal with both the nostril overhang 31a and the septum bulge 31b portions of the nose 30. The shape of the nose buds allow the nose buds to form a tighter seal when the user exhales. Exhalation helps tighten the seal against bottom perimeter of portions of the nostrils and helps prevent a device 12 such as the CPAP from being detached or blown out when the user exhales. Once inserted, device 10 is self-supported by the flanged fittings 16a, 16b, as these flanged fittings are lodged against the nostril overhang 31a and septum bulge 31b.

Referring now to FIG. 4, illustrated is a nose bud estimated centerline angle range. This estimated centerline angle range is about 110 to 150 degrees. This range can be adjustable to an individual's nose for a better fit or it may be that there are several predominant angles that might fit most noses. This estimated centerline angle range is the range of an angle formed between the centerlines of each nose bud. Adjustments could be made for example by allowing flexure of the body 12 about its center line 12′, e.g., via a hinge (not shown) or by properties of the material of the body 12, which would allow the body 12 to be flexed over a range of angles and hold the flexed shape. Other adjustment techniques could be used.

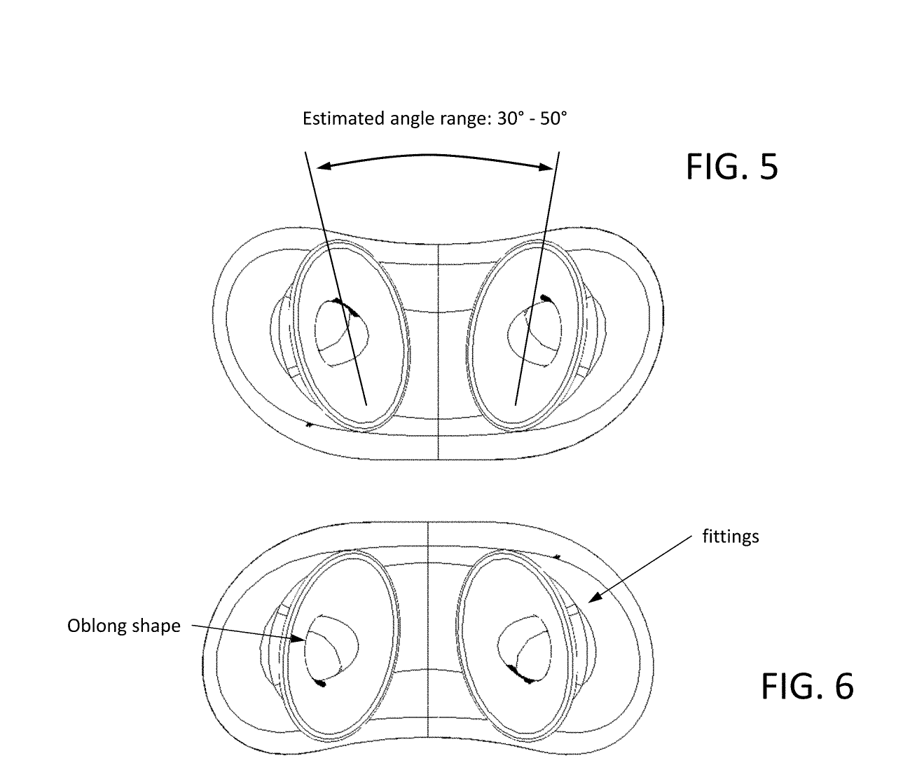

Referring now to FIG. 5, illustrated is the nose bud estimated face angle range of 30-50 degrees. This can be adjusted to the individual nose for better fit or it may be that there are several predominant angles that might fit most noses. This estimated face angle range is the range of an angle formed between planes (or lines) touching external end surfaces of each nose bud. Adjustments could be made for example by allowing flexure of the nose buds about their center lines (not shown) by properties of the material of the nose buds 14a, 14b or by making the nose buds 14a, 14b wall slightly collapsible and repositionable as an accordion, which would allow the nose buds 14a, 14b to be positioned over the range of angles and hold that position. Other adjustment techniques could be used.

Referring now to FIG. 6, illustrated is the nose bud shape as being in some embodiments, generally oblong, so as to approximate common nostril shapes. This can be adjusted to the individual nose for better fit or it may be that there are several predominant shapes that might fit most noses. The nose bud material is compliant, but it is designed to achieve and maintain a certain cross sectional air flow area. The material is a compliant soft durometer material allowing the fittings to conform to nostril shape. Openings can be oblong and can range in an open area, e.g., open surface from about 30-90 mm² for instance.

Materials for the nose buds could be natural and synthetic rubbers or any other material that possesses soft yet compliant properties. Characteristics of such materials include ASTM D2240 Shore A scale 10-70 or the ASTM D2240-00 Shore OO scale 50-100 (ASTM International, PA. USA). Other scales and ranges can be used. Other ranges can be used, for example Shore A scale range can be 10-90 or the ASTM D2240-00 Shore OO scale range can be 30-100. The materials should be hypo-allergic, etc. One suitable material is silicone.

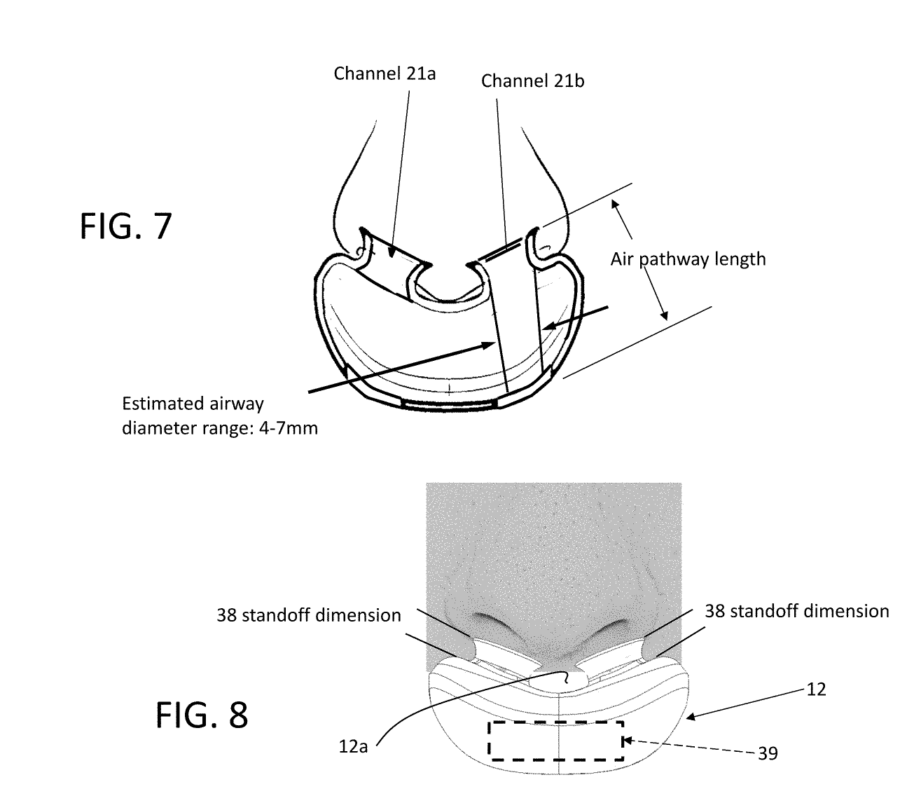

Referring now to FIG. 7, illustrated is the contour of the nose bud inside air pathways designed for minimizing flow restrictions during an exhalation. Smooth inside flared surfaces of the channels 21a, 21b minimize air restrictions (impedance to air flow) upon exhalation. Also shown is the air pathway diameter, e.g., about 4-7 mm, for instance, and length to minimize flow restrictions during an exhalation. The length can be considered as composed of two portions. One portion is the length of the nasal interface, which is based principally on the nose shape and practical applications of the nasal interface can come in various sizes. The other portion is the length through the body 12 of the device 10 the nasal interface attaches to which in this example is the CPAP body 12.

Referring now to FIG. 8, illustrated are nose bud standoff dimensions 38 which control the proper nose bud insertion depth. The range of the standoff dimension 38 is approximately 0.15 to 0.25 inches. Also shown is the top surface 12a of the device body 12 providing a stop against the nose 30 to help position the nose buds and prevent over insertion. Also shown in phantom is a micro pump 39 that is housed within the device body 12. The micro pump 39 as well as other components of the CPAP device 10 is disclosed in the US published patent application US-2015-0267695-A1 incorporated herein by reference. In the published patent the CPAP device with a micro pump has an alternative nasal interface. The alternative nasal interface disclosed in the published application differs from the nasal interface described herein in several aspects as will be set out in the claims.

Elements of different implementations described herein may be combined to form other embodiments not specifically set forth above. Elements may be left out of the structures described herein without adversely affecting their operation. Furthermore, various separate elements may be combined into one or more individual elements to perform the functions described herein.

Other embodiments are within the scope of the following claims. For example, the device body 12 can be of various shapes and can simply have the airway passages that allow a user to breathe in ambient air through the nose bud channels 21a, 21b. In this implementation the device 10 would not include features of the CPAP device such as a micro pump, etc. allowing the device body with nose buds to mitigate snoring by a user.

In some implementations, the device could consist essentially of the nose buds 14a, 14b affixed to a body.

What is claimed is:

1. An airway pressure breathing device comprises:
a body having a pair of air passages through the body, which pair of air passages terminate in a pair of end portions, with each end portion having at least one outlet in a first surface of the end portion;
a micro pump supported by the body, the micro pump configured to pump ambient air through the air passages in the body to the end portions; and
a nasal interface supported on the body, the nasal interface comprising a pair of nose buds having flared fittings at end portions thereof.

2. The airway pressure breathing device of claim 1 wherein the nose buds have nose bud exterior sidewalls, with the flared fittings integrally provided on the nose bud sidewalls, and the flared fittings sized to allow the user to easily insert the nose buds into the user's nose.

3. The airway pressure breathing device of claim 1 wherein the body and nose buds are adjustable to provide a centerline angle and a face angle, respectively within a pair of ranges to provide a seal between the airway pressure breathing device and the user's nose.

4. The airway pressure breathing device of claim 1 wherein the flared fittings have at least one aperture that connects to air pathways in the airway pressure breathing device.

5. The airway pressure breathing device of claim 1 wherein the nose buds have exterior sidewalls having an external mesa shape, with the flared fittings being extensions of the nose bud sidewalls.

6. The airway pressure breathing device of claim 1 wherein the nose buds have exterior sidewalls having an external mesa shape, with the flared fittings being extensions of the nose bud sidewalls, and with the flared fittings having an internal passage way between end portions thereof that is conical-shaped, with a first one of the end portions at the flared portions having a larger opening than a second distal one of the end portions terminating at the airway pressure breathing device.

7. The airway pressure breathing device of claim 1 wherein the material of the nose buds is a supple compliant soft durometer material.

8. The airway pressure breathing device of claim 1 wherein the nose buds have a nose bud estimated centerline angle range of about 110 to 150 degrees, a nose bud estimated face angle range of 30-50 degrees and the nose bud shapes are generally oblong.

9. The airway pressure breathing device of claim 1 wherein the nose buds having openings that are oblong and can range in area from about 30-90 mm².

10. The airway pressure breathing device of claim 1 wherein the material of the nose buds is a rubbery material.

11. A device attachable to a user's nose, the device comprising:
a body having a pair of air passages through the body, which pair of air passages terminate in a pair of end portions, with each end portion having at least one outlet in a first surface of the end portion; and
a nasal interface attached to the body, the nasal interface comprising a pair of nose buds, each nose bud having an external mesa shaped sidewall that terminates at a first end in a flared portion and terminates at the second end at a corresponding one of the pair of air passages of the body.

12. The interface of claim 11 wherein each nose bud sidewall and each corresponding flared fitting are integrally provided with the nose bud sidewalls, and with the flared fittings sized to allow the user to easily insert the nose buds into the user's nose.

13. The interface of claim 11 wherein the nose buds provide a seal between the device and the user's nose.

14. The interface of claim 11 wherein the material of the nose buds is a supple compliant soft durometer material, and with the interface having a nose bud estimated centerline angle range of about 110 to 150 degrees, a nose bud estimated face angle range of 30-50 degrees and the nose bud external sidewall and flared fittings have a shape that is generally mesa shaped.

15. The interface of claim 11 wherein the nose buds have openings there through that are generally oblong and are in a range in area from about 30-90 mm².

16. A nasal interface to attach a device to a user's nose, the interface comprising:
a pair of nose buds each having a sidewall that encloses a passageway, an external shape of the sidewall being mesa-shaped, with the sidewall terminated at one end in a flared fitting and with each of the nose buds having at a second distal opposing end of the sidewall, an accommodation to accept a device.

17. The interface of claim 16 wherein the nose buds each have an internal passage between the flared fitting and the second distal opposing end provided by an internal conical shaped sidewall.

18. The interface of claim 16 wherein the nose buds provide a seal between a device to which the interface is attached and a nostril of the user's nose.

19. The interface of claim 16 wherein the material of the nose buds is a supple compliant soft durometer material, and the interface has a nose bud centerline angle range of about 110 to 150 degrees and a nose bud face angle range of 30-50 degrees.

20. The interface of claim 16 wherein the flared fittings of each nose bud has an opening to the corresponding passageway that is oblong shaped and ranges in area from 30-90 mm².

21. A nasal interface to attach a device to a user's nose, the interface comprising:
a nose bud having a sidewall that encloses a passageway, an external shape of the sidewall being somewhat mesa in shape, with the sidewall terminated at one end in a flared fitting.

22. The interface of claim 21 wherein the nose bud having at a second distal opposing end of the sidewall an accommodation to accept a device.

23. The interface of claim 15 wherein the material of the nose bud is a supple compliant soft durometer material and with the interface having a nose bud estimated centerline angle range of about 110 to 150 degrees, a nose bud estimated face angle range of 30-50 degrees and the nose bud having the passage way being a somewhat funnel shape.

24. The interface of claim 15 wherein interface includes two of the nose buds attached to a device and with each having openings to the passageway that are oblong shaped and range in surface area from about 30-90 mm².
